(19) United States
(12) Patent Application Publication
(10) Pub. No.: US 2019/0060622 A1
(43) Pub. Date: Feb. 28, 2019

(54) CATHETER BALLOON WITH INTEGRATED WIRING

(71) Applicant: Biosense Webster (Israel) Ltd., Yokneam (IL)

(72) Inventor: Christopher Thomas Beeckler, Brea, CA (US)

(21) Appl. No.: 15/684,434

(22) Filed: Aug. 23, 2017

(51) Int. Cl. A61M 29/02

(52) U.S. Cl. CPC A61M 29/02; A61M 2210/125; A61M 2207/10

(57) ABSTRACT

A medical instrument includes a shaft, an inflatable balloon and one or more electrical wires. The shaft is configured for insertion into a body of a patient. The inflatable balloon is coupled to a distal end of the shaft and includes a wall containing an internal cavity. The one or more electrodes are disposed on an external surface of the balloon. The one or more electrical wires connect to the one or more electrodes and run within the wall of the balloon parallel to the external surface.

### FIELD OF THE INVENTION

The present invention relates generally to medical probes, and particularly to design and manufacturing of balloon catheters.

### BACKGROUND OF THE INVENTION

Various known catheter designs have an inflatable balloon fitted at their distal end. For example, U.S. Patent Application Publication 2013/0079614 A1 describes a balloon-tip ablation catheter with an inflatable balloon fitted at its distal side and a high-frequency current distributing electrode and a temperature sensor that are arranged inside the balloon.

As another example, U.S. Pat. No. 8,805,466 describes a tissue electrode assembly that includes a membrane configured to form an expandable, conformable body that is deployable in a patient. The assembly further includes a flexible circuit positioned on a surface of the membrane and comprising at least one base substrate layer, at least one insulating layer and at least one planar conducting layer. An electrically-conductive electrode covers at least a portion of the flexible circuit and a portion of the surface of the membrane not covered by the flexible circuit.

### SUMMARY OF THE INVENTION

An embodiment of the present invention provides a medical instrument including a shaft, an inflatable balloon and one or more electrical wires. The shaft is configured for insertion into a body of a patient. The inflatable balloon is coupled to a distal end of the shaft and includes a wall containing an internal cavity. The one or more electrodes are disposed on an external surface of the balloon. The one or more electrical wires connect to the one or more electrodes and run within the wall of the balloon parallel to the external surface.

In some embodiments, the wires run through one or more lumens that are formed in the wall and run parallel to the external surface. In other embodiments, the lumens are formed by extrusion of the balloon wall.

In an embodiment, the medical instrument further includes one or more temperature sensors, which are located in one or more of the lumens and are electrically connected to one or more integrated wires running through the one or more of the lumens.

In another embodiment, the wires are embedded in the wall material. In an embodiment, the electrical wires are uninsulated. In some embodiments, the electrical wires are electrically connected to electrical wiring running through the shaft. In some embodiments, the one or more electrical wires connect to the one or more electrodes through holes that are formed in the wall through the external surface.

In an embodiment, the electrical wires are glued in place with a conductive adhesive that is injected through the holes. In some embodiments, the electrodes are formed by metallization on the external surface of the balloon, and the metallization extends through the holes so as to create an electrical contact with the electrical wire.

There is additionally provided, in accordance with an embodiment of the present invention, a method for manufacturing a medical instrument. The method includes forming a tube of a polymeric material having a central lumen surrounded by a wall and a plurality of electrical wires running through the wall in a direction parallel to an external surface of the tube. Holes are drilled through the wall to expose the electrical wires to the external surface at selected locations. Electrodes are attached to the external surface of the wall, such that the electrodes contact the electrical wires through the holes in the wall, and a segment of the tube is closed off and expanded so as to form a balloon.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based ablation system, in accordance with an embodiment of the present invention;

FIGS. 2A-F are schematic sectional views showing successive stages in the production of a balloon having electrical wires in lumens running in the wall of the balloon, in accordance with an embodiment of the present invention;

FIGS. 3A-C are schematic sectional views showing successive stages in the production of a balloon having electrical wires embedded in the wall of the balloon, in accordance with another embodiment of the present invention; and

FIG. 4 is a schematic, partly cutaway view of a balloon assembly in an inflated state, in accordance with an embodiment of the present invention.

### DETAILED DESCRIPTION OF EMBODIMENTS

### Overview

Embodiments of the present invention that are described herein provide improved wiring schemes for catheter balloons.

In some embodiments, an inflatable balloon is coupled to a distal end of a shaft, such as the insertion tube of a catheter. Electrodes are disposed on the external surface of the balloon, and electrical wires that are integrated in the wall of the balloon and run parallel to the external surface connect these electrodes to wiring that runs through the shaft.

In the present description and in the claims, integrated wires mean wires running within the wall material of the balloon parallel to the external surface of the balloon. Such integrated wires have no contact with the exterior or interior of the balloon other than through openings made for the purpose of electrical connection, as described below.

In some embodiments, the integration of a wire in the balloon is done by first forming a lumen in the wall material of the balloon, wherein the lumen runs parallel to the wall. The material is processed to expose the lumen at a particular location, and the wire is inserted in the lumen.

In other embodiments, the integration of wires is performed by embedding the wires in the raw material of the balloon, running parallel to the wall, before actually forming the balloon. The material is then processed to extrude the balloon, and to expose the wires at particular locations.

In some embodiments, holes are formed in the external surface of the balloon to expose the embedded wires. Thus, when an electrode is metallized onto the external surface of the balloon in a subsequent manufacturing step, the metallization also forms electrical contact to the embedded wires.

In an embodiment, other electrical devices, such as temperature sensors, are placed in lumens within the wall of the balloon and are connected with the integrated wires.

The disclosed wiring techniques have the advantage, for example, of saving the need to penetrating through the balloon wall in order to connect insulated wiring from the interior lumen of the balloon to devices disposed at its surface. This advantage eliminates potential leakages of pressurized solution that is used to inflate the balloon. This approach results in better safety and reliability of the balloon, while at the same time allowing complex wiring schemes.

Another advantage of the disclosed wiring schemes is that they make it possible to add one or more layers of wiring under the surface of the balloon. For more complicated structures, where the 2D nature of the balloon surface does not allow separate wiring to be connected to devices, such as electrodes, on the balloon surface, the disclosed wiring techniques enable more complex wiring schemes.

Another advantage of the disclosed wiring schemes is that they obviate the need to cement flexible printed circuits to the outside of the balloon, which tend to delaminate after repeated cycling.

Another advantage of the disclosed wiring schemes is that they place the wires flush to the surface of the balloon or recessed. This feature is advantageous as a bump on the exterior surface of the balloon can cause tissue trauma. Moreover, during withdrawal into a sheath such a bump can be subjected to significant friction by the sheath and become delaminated.

Furthermore, embedding wires in the raw material of the balloon or placing wires in lumens extruded within the balloon wall material allows simpler and cheaper balloon manufacturing techniques to be used. For example, embodiments of the present invention make it possible to use uninsulated wires to connect to electrodes on the balloon surface, since the wires are insulated by the wall material of the balloon itself.

### System Description

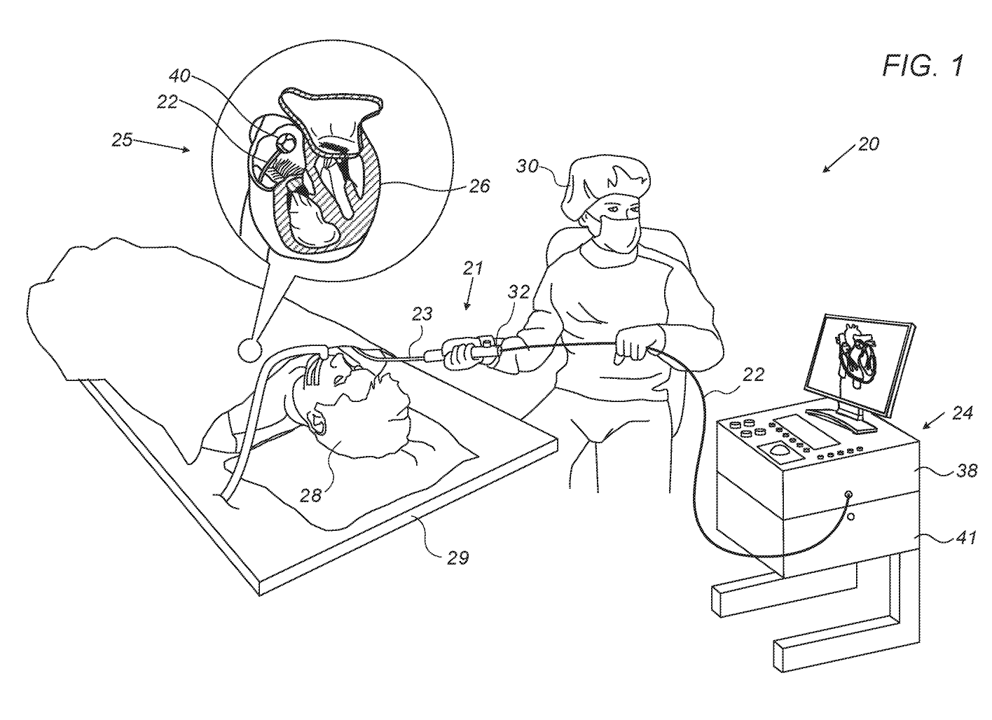

FIG. 1 is a schematic, pictorial illustration of a catheter-based ablation system 20, in accordance with an embodiment of the present invention. System 20 comprises a catheter 21, wherein a shaft 22 of the catheter is inserted into a heart 26 of a patient 28 through a sheath 23. The proximal end of catheter 21 is connected to a control console 24. In the embodiment described herein, catheter 21 may be used for any suitable therapeutic and/or diagnostic purposes, such as electrical sensing and/or ablation of tissue in heart 26.

Console 24 comprises a processor 41, typically a general-purpose computer, with suitable front end and interface circuits 38 for receiving signals from catheter 21, as well as for applying energy via catheter 21 to ablate tissue in heart 26 and for controlling the other components of system 20.

A physician 30 inserts shaft 22 through the vascular system of patient 28 lying on a table 29. Catheter 21 comprises a balloon assembly 40 fitted at the distal end of shaft 22. During the insertion of shaft 22, balloon assembly 40 is maintained in a collapsed configuration. Physician 30 navigates balloon assembly 40 to a target location in heart 26 by manipulating shaft 22 using a manipulator 32 near the proximal end of the catheter. Once the distal end of shaft 22 has reached the target location, physician 30 inflates balloon assembly 40 and operates console 24 so as sense signals and apply ablation energy to the tissue at the target location.

### Balloon with Integrated Wiring

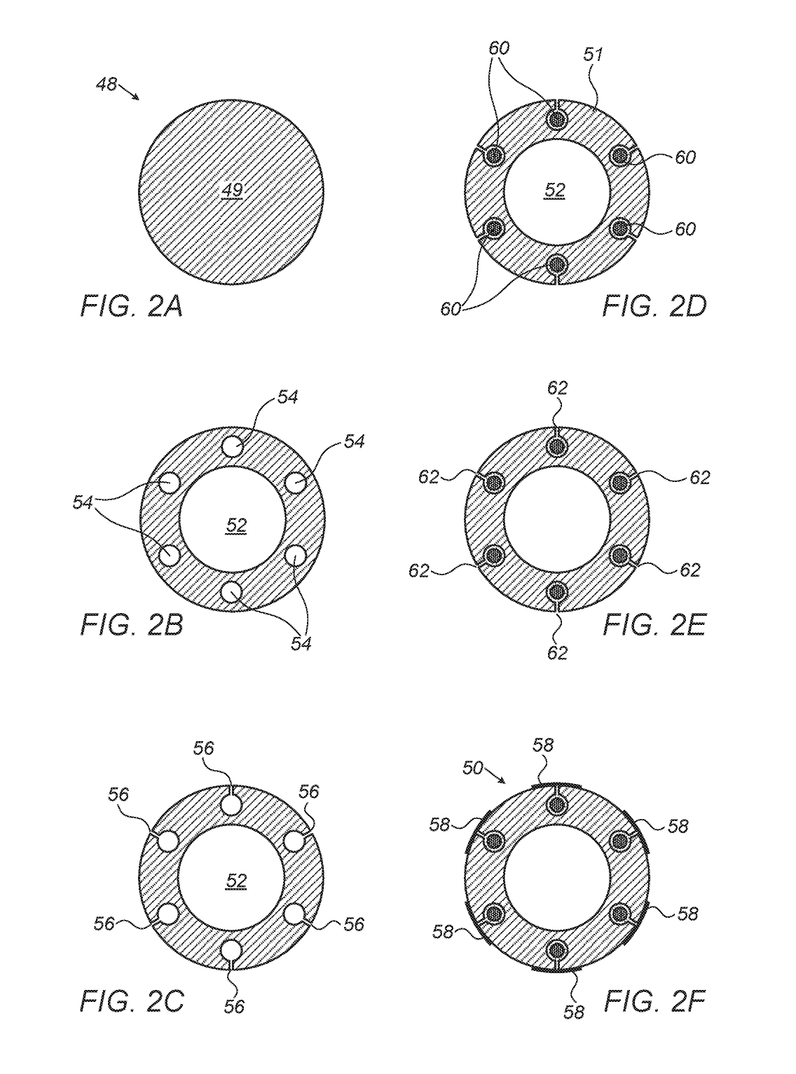

FIGS. 2A-F are schematic sectional views showing successive stages in the production of an inflatable balloon 50, in accordance with an embodiment of the present invention. The manufacturing begins with accepting a cylinder 48 consisting of a homogenous raw material 49, as shown in FIG. 2A. Material 49 typically comprises a biocompatible polymer, for example polyethylene terephthalate (PET), polyurethane, or polyether block amide.

As shown in FIG. 2B, lumens 54 are formed in the periphery of the homogenous cylinder of raw material, for example, by extrusion of the raw material, and running parallel to the external surface of the cylinder. At the same processing step, a central lumen 52 is also formed, which will subsequently become the cavity of the finished balloon. Balloon 50 now contains a wall 51 with small lumens 54 extending through the wall. Small lumens 54 may be filled with sacrificial mandrels (not shown) which can be removed after forming the balloon.

A segment of the tubular extruded wall material is then cut to the appropriate length, closed off at the distal end, and blown into a balloon, or formed by any other suitable production method (processing steps not shown).

As shown in FIG. 2C, small holes 56 are drilled into the formed balloon, for example using a laser or by mechanical means, to expose lumens 54. Holes 56 may have any form or combination of forms, for example slot-shaped or round.

As shown in FIG. 2D, electrical wires 60, which need not be insulated, are threaded through some or all of lumens 54, possibly before the segment is blown into a balloon. Optionally, the wires are glued in place with a conductive adhesive, such as a suitable epoxy 62, which is injected through holes 56, as shown in FIG. 2E.

After the wires have been glued, the exterior surface of balloon 50 is metallized to form electrodes 58 on the surface, as shown in FIG. 2F. The metallization can be carried out using any suitable technique that is known in the art, for example sputtering, plating, or any other form of Physical Vapor Deposition (PVD). The metal of electrodes 58 will adhere to the surface of the balloon and to epoxy 62, thus making electrical contact with wires 60. Alternatively, if epoxy 62 is not used, the metallization itself can extend through holes 56 into lumens 54 to create an electrical contact between wires 60 and the exterior metallization, while remaining level with the surface.

In some embodiments, additional devices can be placed in lumens 54, for example, thermocouples (not shown).

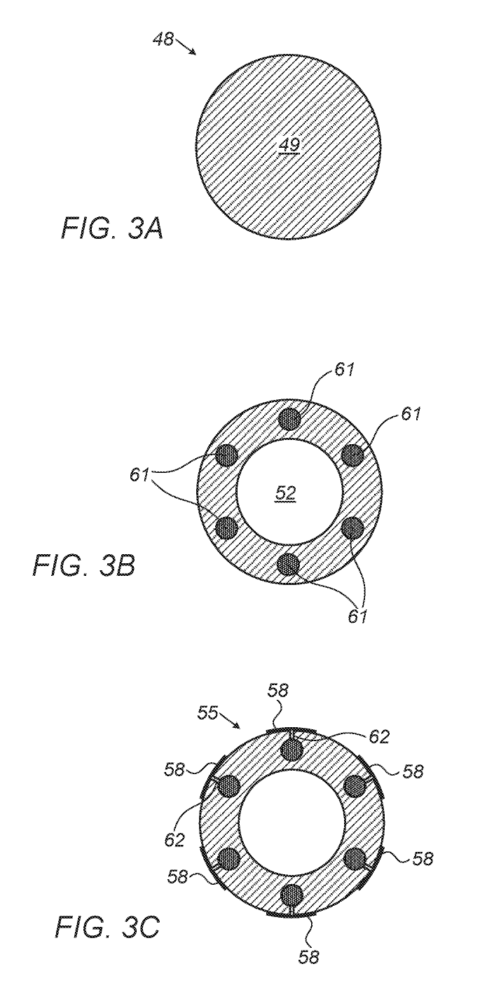

FIGS. 3A-C are schematic sectional views showing successive stages in the production of another inflatable balloon 55, in accordance with an alternative embodiment of the invention. The manufacturing begins with accepting a cylinder 48 consisting of a homogenous raw material 49, as shown in FIG. 3A. A plurality of wires 61 peripherally are embedded in material 49 during the extrusion process, running in a direction parallel to an external surface of the cylinder. At the same extrusion process, cylinder 48 is extruded to form central lumen 52, as illustrated in FIG. 3B.

Subsequent manufacturing steps resemble those shown and described above in reference to the preceding embodiment:

a) Central lumen 52 is formed;

b) Holes 56 are drilled to expose wires 61;

c) Conductive epoxy 62 is injected through holes 56; and

d) The exterior surface of balloon 55 is metallized to form electrodes 58 on the surface.

The result of the above steps is shown in FIG. 3C.

Alternatively, as noted above, the metallization itself can extend through holes 56 into lumens 54 to create an electrical contact between wires 61 and the exterior metallization, while remaining level with the surface.

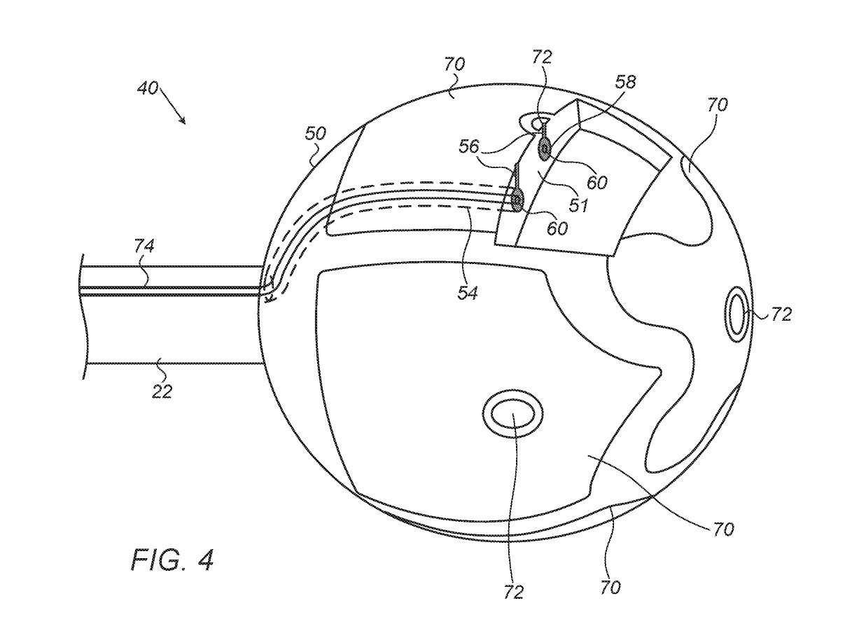

FIG. 4 is a schematic pictorial illustration of balloon assembly 40, following production of balloon 50 as described above. Balloon 50 is shown in an inflated state, connected to the distal end of shaft 22. Electrodes 70, 72 are disposed over the external surface of balloon 50, including ablation electrodes 70 and sensing electrodes 72, which are connected to wires 60 running parallel to the external surface of the balloon within wall 51. Ablation electrode 70 and sensing electrode 72 are in electric contact with wires 60, which are glued inside lumens 54 in wall 51, through holes 56. Wires 60 are further electrically connected at their proximal ends to additional electrical wiring 74 running through shaft 22 to console 24. Although sensing electrodes 72 are configured as “islands” within the larger ablation electrodes 70, integration of wires 60 within wall 51 makes it possible to make electrical connections to both electrodes 70 and 72 without requiring a complicated printed circuit topology on the balloon surface or running wires through the internal cavity of balloon 50.

The example configurations shown in the figures are chosen purely for the sake of conceptual clarity. In alternative embodiments, the disclosed techniques may use other suitable configuration of wiring. For example, any number and arrangement of lumens 54, wires 60 and/or wires 61, and holes 56 is possible.

Moreover, the disclosed techniques are not limited to balloon assemblies, and can be used with other suitable distal-end assemblies that comprise electrical devices.

Although the embodiments described herein mainly address balloon assemblies for cardiac applications, the methods and systems described herein can also be used in other medical applications.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
a shaft for insertion into a body of a patient;
an inflatable balloon coupled to a distal end of the shaft and comprising a wall containing an internal cavity;
one or more electrodes disposed on an external surface of the balloon; and
one or more electrical wires, which connect to the one or more electrodes and run within the wall of the balloon parallel to the external surface.

2. The medical instrument according to claim 1, wherein the wires run through one or more lumens that are formed in the wall and run parallel to the external surface.

3. The medical instrument according to claim 2, wherein the lumens are formed by extrusion of the balloon wall.

4. The medical instrument according to claim 2, and comprising one or more temperature sensors, which are located in one or more of the lumens and are electrically connected to one or more integrated wires running through the one or more of the lumens.

5. The medical instrument according to claim 1, wherein the wires are embedded in the wall material.

6. The medical instrument according to claim 1, where the electrical wires are uninsulated.

7. The medical instrument according to claim 1, wherein the electrical wires are electrically connected to electrical wiring running through the shaft.

8. The medical instrument according to claim 1, wherein the one or more electrical wires connect to the one or more electrodes through holes that are formed in the wall through the external surface.

9. The medical instrument according to claim 8, wherein the electrical wires are glued in place with a conductive adhesive that is injected through the holes.

10. The medical instrument according to claim 8, wherein the electrodes are formed by metallization on the external surface of the balloon, and wherein the metallization extends through the holes so as to create an electrical contact with the electrical wire.

11. A method for manufacturing a medical instrument, the method comprising:
forming a tube of a polymeric material having a central lumen surrounded by a wall and a plurality of electrical wires running through the wall in a direction parallel to an external surface of the tube;
drilling holes through the wall to expose the electrical wires to the external surface at selected locations;
attaching electrodes to the external surface of the wall, such that the electrodes contact the electrical wires through the holes in the wall; and
closing off and expanding a segment of the tube so as to form a balloon.

12. The method according to claim 11, wherein forming the tube comprises extruding the polymeric material to form the central lumen and a plurality of peripheral lumens running through the wall in the direction parallel to the external surface of the tube, and threading the electrical wires through the peripheral lumens.

13. The method according to claim 11, wherein forming the tube comprises embedding the electrical wires in a cylinder of the polymeric material, and extruding the cylinder to form the central lumen in the tube.

14. The method according to claim 11, wherein the electrical wires are uninsulated.

15. The method according to claim 11, and comprising injecting a conductive adhesive through the holes so as to glue the electrical wires in place.

16. The method according to claim 11, wherein attaching the electrodes comprises metallizing the electrodes onto the external surface of the balloon, wherein metallization extends through the hole so as to create an electrical contact with the electrical wires.

17. The medical instrument according to claim 11, wherein the holes are drilled through the wall of the balloon by a laser.

18. The medical instrument according to claim 11, wherein the holes are mechanically drilled through the wall.
